(19) United States
(12) Patent Application Publication
(10) Pub. No.: US 2011/0206773 A1
(43) Pub. Date: Aug. 25, 2011

(54) SUSTAINED DELIVERY OF DRUGS FROM BIODEGRADABLE POLYMERIC MICROPARTICLES

(75) Inventors: Erin Lavik, Cleveland Heights, OH (US); James Bertram, New Haven, CT (US); Sandeep Saluja, Fair Lawn, NJ (US); Markus Kuehn, Amana, IA (US); Young H. Kwon, Iowa City, IA (US); Rebecca Robinson, East Point, GA (US); John J. Huang, Hamden, CT (US)

(73) Assignees: Yale University; University of Iowa Research Foundation

(21) Appl. No.: 12/945,246

(22) Filed: Nov. 12, 2010

Related U.S. Application Data
(63) Continuation-in-part of application No. PCT/US2009/044732, filed on May 20, 2009.
(60) Provisional application No. 61/054,506, filed on May 20, 2008.
(60) Provisional application No. 61/054,511, filed on May 20, 2008.
(60) Provisional application No. 61/054,519, filed on May 20, 2008.
(60) Provisional application No. 61/260,522, filed on Nov. 12, 2009.

(51) Int. Cl. A61K 9/14; A61K 31/573; A61K 31/216; A61K 31/517; A61P 27/02; B65D 69/00

(52) U.S. Cl. 424/501; 424/489; 514/179; 514/180; 514/530; 514/266.4; 206/223

(57) ABSTRACT

Biodegradable polymeric microparticle compositions containing one or more active agents, especially those useful for treating or preventing or one or more diseases or disorders of the eye, and methods of making and using thereof, are described. The microsphere compositions release an effective amount of the one or more active agents for a period greater than 14 days in vivo, preferably greater than 60 days in vivo, more preferably up to 73 days in vivo, more preferably greater than 90 days in vivo, even more preferably over 100 days in vivo, and most preferably greater than 107 days in vivo. In a preferred embodiment, the microparticle compositions contain one or more active agents such as AG1478 to induce nerve regeneration, specifically regeneration of the optic nerve useful for managing elevated intraocular pressure (TOP) in the eye.

### CROSS-REFERENCE TO RELATED APPLICATIONS

This application is a continuation-in-part of International Application No. PCT/US2009/044732 entitled “Sustained Delivery of Drugs from Biodegradable Polymeric Microparticles”, filed on May 20, 2009, which claims priority to U.S. Ser. No. 61/054,519 entitled “Sustained Delivery of Ofloxacin, Prednisolone Acetate, and Methotrexate from Polylactic-co-glycolic acid) Microspheres”, filed on May 20, 2008; U.S. Ser. No. 61/054,511 entitled “Sustained Delivery of Travoprost to Lower Intraocular Pressure”, filed on May 20, 2008; and U.S. Ser. No. 61/054,506 entitled “Sustained Delivery of AG 1478, An Inhibitor of the Epidermal Growth Factor Receptor (EGFR), for Antitumor Therapy and Neural Regeneration”, filed on May 20, 2008. This application also claims priority to U.S. Ser. No. 61/260,522 entitled “Sustained Delivery of Drugs from Biodegradable Polymeric Microparticles”, filed on Nov. 12, 2009. The disclosures in the applications listed above are herein incorporated by reference.

### FIELD OF THE INVENTION

This invention is in the field of pharmaceutical compositions comprising biodegradable microparticles encapsulating high weight percent drug and providing sustained release over a prolonged period of time of drug levels bioequivalent to direct administration of drug and methods of use thereof.

### BACKGROUND OF THE INVENTION

Polymeric microparticles have been used for drug delivery for decades. Numerous methods to increase the amount of drug which can be delivered, and to manipulate rate of release, and release profile, have been described. Methods have included altering microparticle size, shape, polymer composition, inclusion of additives such as surfactants and pore forming agents, and inclusion of ligands and bioadhesive agents.

Glaucoma is an ophthalmic disease characterized by the gradual degeneration of retinal ganglion cells (RGCs). RGCs synapse with bipolar cells and transmit visual inputs to the brain along the optic nerve. Degeneration of these cells leads to gradual vision loss and ultimately blindness if untreated. Glaucoma is the second leading cause of blindness (Biomdahl et al., Acta. Opth. Scan., 75, 310-319 (1997)). Glaucoma will affect approximately 60.5 million people in 2010, increasing to 796 million people in 2020 (Quigley et al., Brit. J. Opth., 90, 262-267 (2006)). This includes peoples suffering from both open angle (OAG) and angle closure glaucoma (ACG).

Although a normal tension variant does exist, the development of glaucoma is most often associated with elevated intraocular pressure (IOP) (Migdal et al., Opthmal., 101, 1651-1656 (1994)). This elevated pressure is caused by an excess accumulation of aqueous humor in the eye due to blockage of the trabecular network (Alward et al., Amer. J. Opthmal., 126, 498-505 (1998)). With a majority of glaucoma cases associated with elevated IOP, reduction of this pressure has been found to greatly mitigate degeneration in approximately 90% of the cases, including cases in which IOP is in the normal range but optic neuropathy occurs (Id.).

Eye drops containing one more active agents that lower IOP are typically prescribed to treat glaucoma. Eye drops are currently the primary means of delivery for this drug. However, eye drop typically deliver very small amounts of drug, requiring large numbers of doses per day for TOP management. Compliance with this treatment regime is poor with more than half of patients unable to maintain consistently lowered kW through drops (Rotchford and Murphy, Brit. J. Opthmal., 12, 234-236 (1998)).

Drops also lead to extensive systemic absorption of the administered drug (˜80%, Marquis and Whitson, Drugs & Aging, 22, 1-21 (2005)). This systemic absorption can result in adverse side effects. Together, these complications make topical application of IOP-lowering drugs problematic, especially in the aging population that exhibits the lowest compliance and highest degree of complications (Marquis and Whitson, Drugs & Aging, 22, 1-21 (2005)). There exists a need for sustained release formulations, which overcomes the limitations of currently available eye drops. There also exists a need for sustained release formulations and methods of use thereof that promote nerve regeneration in patients suffering from glaucoma.

A variety of approaches for the sustained delivery of drugs have been investigated

U.S. Pat. No. 6,726,918 to Wong describes methods for treating inflammation-mediated conditions of the eye, the methods including implanting into the vitreous of the eye a bioerodible implant containing a steroidal anti-inflammatory and a bioerodible polymer, wherein the implant delivers an agent to the vitreous in amount sufficient to reach a concentration equivalent to at least about 0.65 μg/ml dexamethasone within about 48 hours and maintains a concentration equivalent to at least about 0.03 μg/ml dexamethasone for at least about three weeks. Wong does not disclose administering the implants by subconjunctive injection. Wong does not disclose formulations which provide sustained release of an effective amount of the drug for several weeks to months. Wong does not disclose or suggest compositions or methods of use thereof that promote optical nerve regeneration.

U.S. Patent Application Publication No. 2006/0173060 to Chang et al. describes biocompatible microparticles containing an alpha-2-adrenergic receptor agonist and a biodegradable polymer. The microparticles can allegedly be used to treat glaucoma. Chang alleges that the microparticles release the active agent for a period of time of at least about one week, such as between two and six months. Chang discloses that the microparticles can be administered subconjunctivally. Chang does not disclose or suggest compositions or methods of use thereof that promote optical nerve regeneration.

U.S. Patent Application Publication No. 2004/0234611 to Ahlheim et al. describes an ophthalmic depot formulation containing an active agent embedded in a pharmacologically acceptable biocompatible polymer or a lipid encapsulating agent for periocular or subconjunctival administration. The formulation can be in the form of microparticles or nanoparticles. Ahlheim discloses that the depot formulations are adapted to release all or substantially all of the active material over an extended period of time (e.g., several weeks up to 6 months). Suitable active agents are listed in paragraphs 0033 to 0051; however, the preferred active agent is a staurosporine, a phthalazine, or a pharmaceutically salt thereof. Suitable polymers are listed in paragraphs 0014 to 0026. Ahlheim contains no examples showing in vitro or in vivo release of any active agents. Ahlheim does not disclose or suggest compositions or methods of use thereof that promote optical nerve regeneration.

None of the references discussed above disclose optimizing the charge, hydrophilicity or hydrophobicity, and/or the molecular weight of the polymers used to prepare the microparticles in order to maximize drug loading and release of an effective amount of the drug for a desired period of time.

Therefore, it is an object of the invention to provide sustained release polymeric microparticulate compositions which have been optimized to maximize drug loading and release an effective amount of a drug (or drugs) for a desired period of time.

It is a further object of the present invention to provide such formulations useful for reducing intraocular pressure (IOP) which provide sustained release of an amount of drug comparable to that administered topically for more than 14 days in vivo, and methods of making and using thereof.

It is further an object of the invention to provide sustained release compositions of one or more active agents useful for reducing intraocular pressure (IOP) which provide sustained release for more than 14 days in vivo, and methods using thereof, wherein the compositions exhibit minimal adverse side effects and is well tolerated by patients.

It is still further an object of the invention to provide sustained release compositions of one or more active agents useful for promoting regeneration of the optic nerve and methods of making and using thereof.

### SUMMARY OF THE INVENTION

Biodegradable polymeric microparticle compositions containing one or more poorly water soluble active agents, especially those useful for promoting nerve regrowth, and methods of making and using thereof, are described. The microparticles are optimized for the drug to be delivered, so that the hydrophobicity, or hydrophilicity of the polymer and charge of the polymer maximizes loading of the drug, and the selection and molecular weight of the polymers maximize release of an effective amount of the drug for the desired period of time. For example, poorly water soluble drugs tend to interact more strongly with hydrophobic monomers or polymers.

In a preferred embodiment, the microparticle compositions contain one or more active agents such as AG1478 to induce nerve regeneration, specifically regeneration of the optic nerve useful for managing elevated intraocular pressure (IOP) in the eye. The microsphere compositions release an effective amount of the one or more active agents for a period greater than 14 days in vivo, preferably greater than 30 days in vivo, preferably greater than 60 days in vivo, more preferably up to 73 days in vivo, more preferably greater than 90 days in vivo, even more preferably over 100 days in vivo, and most preferably greater than 120 days in vivo. In some embodiments, release of an effective amount is achieved in vivo for periods greater than 150 days, 180 days, 200 days, 250 days, or 270 days.

The desired amount and duration of release is dependent upon several factors including the disease or disorder to be treated, the one or more active agents to be delivered, and the frequency of administration. In one embodiment, the drug is released over a shorter period of time, for example, 14-21 days for steroids such as prednisolone. Alternatively, for delivery of agents to promote neural regeneration, the formulations preferably release drug over longer time periods. In another embodiment, the microparticles may contain two or more drugs in which one or more of the drugs are released over a short amount of time, e.g., 14-21 days to treat an acute condition while one or more drugs are released over an extended period of time, e.g. several weeks to several months to treat a chronic condition, such as nerve degeneration.

In one embodiment, the microspheres are formed from polylactide-co-glycolide (“PLGA”). In another embodiment, the microspheres are formed from a blend of PLGA and polylactic acid (“PLA”). Higher molecular weight polymers, having different ratios of lactic acid (“LA”) (which has a longer degradation time, up to one to two years) to glycolic acid (“GA”) (which has a short degradation time, as short as a few days to a week), are used to provide release over a longer period of time. The combination of drug loading and release rate, as well as the minimization of initial burst release, result in prolonged release of a higher amount of drug. As demonstrated by the examples, the microsphere compositions release a water insoluble drug for at least 35 days, preferably for at least 50 days, more preferably for at least 75 days, most preferably for at least 100 days. The sustained release of drug, in combination with the ability to administer the drug in a minimally invasive manner, should increase patient compliance.

The percent loading of the drug in the microspheres is from about 1% to about 80% by weight, preferably from about 1% to about 60% by weight, more preferably from about 1% to about 40% by weight, more preferably from about 1% to about 25% by weight, more preferably from about 1% to about 20% by weight, most preferably from about 1% to about 10%. The percent loading is dependent on the drug to be encapsulated, the polymer or polymers used to form the microparticles, and/or the procedure used to prepare the microparticles.

In another embodiment, the encapsulation efficiency is between about 50% and about 80%, preferably from about 55% to about 80%, more preferably from about 55% to about 75%, most preferably from about 65% to about 75%. In a particular embodiment, the encapsulation efficiency is about 55%, about 65%, or about 75%.

The microsphere compositions can be administered to the eye using a variety of techniques in the art. In one embodiment, the compositions are administered to the eye by injection. In a preferred embodiment, the microsphere composition is administered subconjunctivally. Subconjunctival administration is minimally invasive, and minimizes systemic absorption of the active agents.

The compositions can be used to treat a variety of diseases or disorders of the eye. Examples of diseases or disorders to be treated include glaucoma, uveitis, post surgical ocular inflammation and/or infection, dry eye syndrome, and macular degeneration. In one embodiment, the compositions are administered to promote nerve regeneration, such as optical nerve regeneration, in patients in need thereof.

### BRIEF DESCRIPTION OF THE DRAWINGS

FIG. 1 is a graph showing the cumulative prednisolone acetate release (μg/mg of polymer) in vitro as a function of time (days) for microparticles prepared from PLGA 502H (▪), PLGA 503H (x), and a blend of PLGA 50211 and PLA (▾).

FIG. 2 is a graph showing the cumulative prednisolone acetate release (μg/mg of polymer) in vitro as a function of time (days) for microparticles prepared from PLGA 50211 (▪), PLGA 50211 with PEG-1500 (▴), PLGA 50211 with sonication (15% amp) (▾), PLGA 50211 with sonication (30% amp) (♦), PLGA 502H with sonication (38% amp) (), and PLGA 502H with homogenizer (□).

FIG. 3 is a graph showing the in vitro cumulative methotrexate release (μg/mg) as a function of time (days) from microspheres prepared from PLGA 503H.

FIG. 4 are graphs showing the in vitro and in vivo cumulative methotrexate release (μg/mg) as a function of time (days) from microspheres prepared from PLGA 502H.

FIG. 5 is a graph showing the release of triamcinolone (μg/mg) in vivo and in vivo as a function of time (days) from PLGA 50211 microparticles.

FIG. 6 is a graph showing the in vitro release profile of travoprost (μg drug/mg polymer) as a function of time (days) for travoprost-loaded PLGA 50311 microparticles.

FIG. 7 is a graph showing the in vitro release of AG1478 (μg drug/mg polymer) as a function of time (days) from PLGA 50311 microparticles.

FIG. 8 is a graph showing the cumulative release of AG1478 (μg/mg polymer) in vitro from microspheres prepared from PLGA 50311 via an oil-in-water emulsion technique, wherein percent drug loading is 2.5% (□) and 5.0% (▴).

FIG. 9 is a graph showing the in vitro release profile of AG1478 (μg drug/mg polymer) as a function of time (days) for AG1478-loaded PLGA 503H microparticles prepared using a S/O/W emulsion process (x), a 0/W emulsion process (□), and a 0/W ea-solvent emulsion process ().

FIG. 10 is a graph showing the cumulative release of AG1478 (μg/mg polymer) in vitro as a function of time (days) from microspheres prepared from PLGA 503H (), PLGA 504 (⋄), and PLGA 504H (□) using an oil-in-water emulsion cosolvent technique.

FIG. 11 is a graph showing the cumulative release of AG1478 (μg/mg polymer) in vitro as a function of time (days) from microspheres prepared from PLGA 504 (⋄) and PLGA 504H (□) prepared using an oil-in-water cosolvent technique.

FIG. 12 is a graph showing cumulative release of AG1478 (μg drug/mg polymer) as a function of time (days) for AG1478 microspheres. Experiment performed in triplicate. Mean±SD

### DETAILED DESCRIPTION OF THE INVENTION

### I. Definitions

“Nanoparticle”, as used herein, refers to particle or a structure in the nanometer (nm) range, typically from about 1 nm to about 1000 nm in diameter, which is encapsulated within the polymer.

“Microparticle”, as used herein, unless otherwise specified, generally refers to a particle of a relatively small size, but not necessarily in the micron size range; the term is used in reference to particles of sizes that can be, for example, administered to the eye subconjunctivally, and thus can be less than 50 nm to 100 microns or greater. Microparticles specifically refers to particles having a diameter from about 1 to about 25 microns, preferably from about 10 to about 25 microns, more preferably from about 10 to about 20 microns. In one embodiment, the particles have a diameter from about 1 to about 10 microns, preferably from about 1 to about 5 microns, more preferably from about 2 to about 5 microns. As used herein, the microparticle encompasses microspheres, microcapsules and microparticles, unless specified otherwise. The relative sizes of microparticles and nanoparticles in the context of the present invention are such that the latter can be incorporated into the former. A micro- or nanoparticle may be of composite construction and is not necessarily a pure substance; it may be spherical or any other shape.

Formulations can be prepared using a pharmaceutically acceptable “carrier” composed of materials that are considered safe and effective and may be administered to an individual without causing undesirable biological side effects or unwanted interactions. As used herein, the “carrier” is all components present in the pharmaceutical formulation other than the active ingredient or ingredients. The term “carrier” includes, but is not limited to, solvents, suspending agents, dispersants, buffers, pH modifying agents, isotonicity modifying agents, preservatives, antimicrobial agents, and combinations thereof.

“Poorly water soluble drug”, as used herein, refers to a drug having a solubility of less than 10 mg/ml at 25° C., preferably less than 5 mg/ml at 25° C., more preferably less than 1 mg/ml at 25° C., most preferably less than 0.5 mg/ml at 25° C.

“Water-soluble drug”, as used herein, refers to a drug having a solubility of greater than 10 mg/ml at 25° C., preferably greater than 25 mg/ml at 25° C., more preferably greater than 50 mg/ml at 25° C., most preferably greater than 100 mg/ml at 25° C.

“Hydrophilic polymer”, as used herein, refers to polymers that have an affinity for water, though are not water soluble.

“Hydrophobic polymer”, as used herein, refers to polymers that tend to repel water.

### II. Compositions

A. Active Agents

The microparticle compositions described herein contain one or more poorly water soluble active agents. In one embodiment, the one or more active agents are useful for treating diseases or disorders of the eye. Suitable classes of active agents include, but are not limited to, active agents that lower intraocular pressure, antibiotics, anti-inflammatory agents, chemotherapeutic agents, agents that promote nerve regeneration, steroids, and combinations thereof. The active agents described above can be administered alone or in combination to treat diseases or disorders of the eyes.

Alternatively, the poorly water soluble drug can be co-administered with a water-soluble drug, either in the same microspheres or in different microspheres or microparticles. The water-soluble drugs can be formulated in polymeric microparticles in which the hydrophilicity, molecular weight, and/or monomer composition has been optimized to maximize loading of the drug in the microparticles and to provide sustained release for a period greater than 14 days in vivo, preferably greater than 30 days, preferably greater than 60 days in vivo, more preferably up to 73 days in vivo, more preferably greater than 90 days in vivo, even more preferably over 100 days in viva, and most preferably greater than 120 days in viva, most preferably at least 175 days in vivo. Microparticles containing water-soluble active agents, and methods of making and using thereof, are described in WO 2008/157614.

Active Agents that Lower IOP

In one embodiment, the microparticles contain one or more active agents that manage (e.g., reduce) elevated LOP in the eye. Suitable active agents include, but are not limited to, prostaglandins analogs, such as travoprost, bimatoprost, latanoprost, unoprostine, and combinations thereof; and carbonic anhydrase inhibitors (CAL), such as methazolamide, and 5-acylimino- and related imino-substituted analogs of methazolamide; and combinations thereof. The microparticles can be administered alone or in combination with microparticles containing a second drug that lowers LOP. The second drug can be poorly water soluble or water-soluble and can be formulated in the same microparticles or different microparticles as described above.

Antibiotics

The microparticles compositions can contain one or more poorly water soluble antibiotics. Exemplary antibiotics include, but are not limited to, cephaloridine, cefamandole, cefamandole nafate, cefazolin, cefoxitin, cephacetrile sodium, cephalexin, cephaloglycin, cephalosporin C, cephalothin, cafcillin, cephamycins, cephapirin sodium, cephradine, penicillin BT, penicillin N, penicillin O, phenethicillin potassium, pivampic ulin, amoxicillin, ampicillin, cefatoxin, cefotaxime, moxalactam, cefoperazone, cefsulodin, ceflizoxime, ceforanide, cefiaxone, ceftazidime, thienamycin, N-formimidoyl thienamycin, clavulanic acid, penemcarboxylic acid, piperacillin, sulbactam, cyclosporins, and combinations thereof.

Inhibitors of Growth Factor Receptors

In another embodiment, the poorly water soluble active agent is an inhibitor of a growth factor receptor. Suitable inhibitors include, but are not limited to, inhibitors of Epidermal Growth Factor Receptor (EGFR), such as AG1478, and EGFR kinase inhibitors, such as BIBW 2992, erlotinib, gefitinib, lapatinib, and vandetanib.

AG1478 is a potent inhibitor of the epidermal growth factor receptor (EGFR). It was developed initially as a small-molecule tyrosine kinase antagonist to treat tumors, such as breast and ovarian tumors, having large excesses of EGFR on their surfaces. EGFR is present in many cell types in the body and is responsible for mediating basic cell behaviors such as proliferation and fate choice of cells, thus making systemic knockdown of EGFR problematic.

To treat a tumor, one would like to have a delivery technology that provides a large and sustained dose of the drug in a localized manner. By fabricating microspheres that deliver AG1478 for an extended period of time (e.g., over 3 months and up to 9 months), one has an injectable technology that can be delivered in a minimally invasive manner, and can deliver the drug over a long period of time in a localized manner. This will allow higher concentrations of the drug at the tumor over longer time periods and limited amounts at other sites potentially augmenting the therapeutic effect of the drug and mitigating side effects.

AG1478 has also been shown to have a role in neural regeneration. By delivering AG1478 in a sustained and localized manner, it may be possible to promote regeneration following injury to the central nervous system (CNS).

Chemotherapeutic Agents and Steroids

The microparticle compositions can contain one or more poorly water soluble chemotherapeutic agents and/or steroids. In one embodiment, the poorly water soluble chemotherapeutic agent is methotrexate. Methotrexate is an antimetabolite which has been used to treat autoimmune disorders as well as certain types of cancers. In the eye, methotrexate is used to treat a number of inflammatory diseases, such as uveitis. Methotrexate is known to cause adverse side effects when administered systemically. Sustained, local delivery has the potential to reduce the amount of methotrexate in serum or eliminate it completely and thus mitigate adverse side effects.

In another embodiment, the drug is a poorly water soluble steroid, such as prednisolone acetate, triamcinolone, prednisolone, hydrocortisone, hydrocortisone acetate, hydrocortisone valerate, vidarabine, fluorometholone, fluocinolone acetonide, triamcinolone acetonide, dexamethasone, dexamethasone acetate, and combinations thereof.

The side effects most associate with ophthalmic surgery are post-operative ocular inflammation and/or infection. Topical administration of eye drops containing a steroid and an antibiotic has typically been used for controlling inflammation and preventing infection. However, poor patient compliance and/or the risk of re-opening the stitched wound due to continuous touching of the wound when applying the drops are limitations of such formulations. Therefore, it is preferable to use sustained release formulations that release the steroid and/or antibiotic over extended periods of time (e.g., 2-3 weeks) to minimize dosing frequency, improve compliance, reduce side effects, and keep the stitched wound intact. Triamcinolone is a steroid used to treat macular odeama, a complication of diabetes and retinal vein occlusion.

Pharmaceutically Acceptable Salts

The one or more active agents can be administered as the free acid or base or as a pharmaceutically acceptable acid addition or base addition salt.

Examples of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalenesulfonic, methanesulfonic, ethane disulfonic, oxalic, and isethionic salts.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

B. Polymers

The microparticles described here can be formed from natural and/or synthetic polymeric materials. “Polymer” or “polymeric”, as used herein, refers to oligomers, adducts, homopolymers, random copolymers, pseudo-copolymers, statistical copolymers, alternating copolymers, periodic copolymer, bipolymers, terpolymers, quaterpolymers, other forms of copolymers, substituted derivatives thereof, and combinations of two or more thereof (i.e., polymer blends). The polymers can be linear, branched, block, graft, monodisperse, polydisperse, regular, irregular, tactic, isotactic, syndiotactic, stereoregular, atactic, stereoblock, single-strand, double-strand, star, comb, brush, dendritic, and/or ionomeric.

Bioerodible polymers may be used, so long as they are biocompatible. Preferred bio-erodible polymers are polyhydroxyacids such as polylactic acid and copolymers thereof. These are approved for implantation into humans. Another class of approved biodegradable polymers is the polyhydroxyalkanoates.

Other suitable polymers are known in the art. They include, but are not limited to: polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene polyethylene glycol), poly(ethylene oxide), poly(ethylene terephthalate), poly(vinyl alcohols), poly(vinyl acetate), poly vinyl chloride polystyrene and polyvinylpryrrolidone.

The bioerodable polymers may be used to form nanoparticles or microparticles which provide delayed or extended release a diagnostic, therapeutic, or prophylactic agent.

As demonstrated by the examples, the percent loading is increased by “matching” the hydrophilicity or hydrophobicity of the polymer to the agent to be encapsulated. In some cases, such as PLGA, this can be achieved by selecting the monomer ratios so that the copolymer is more hydrophilic for hydrophilic drugs or less hydrophilic for hydrophobic drugs. Alternatively, the polymer can be made more hydrophilic, for example, by introducing carboxyl groups onto the polymer. A combination of a hydrophilic drug and a hydrophobic drug can be encapsulated in microparticles prepared from a blend of a more hydrophilic PLGA and a hydrophobic polymer, such as PLA.

The percent loading of the drug in the microspheres is from about 1% to about 80% by weight, preferably from about 1% to about 60% by weight, more preferably from about 1% to about 40% by weight, more preferably from about 1% to about 25% by weight, more preferably from about 1% to about 20% by weight, most preferably from about 1% to about 10%. The percent loading is dependent on the drug to be encapsulated, the polymer or polymers used to form the microparticles, and/or the procedure used to prepare the microparticles.

The preferred polymer is a PLGA copolymer or a blend of PLGA and PLA. The molecular weight of PLGA is from about 10 kD to about 80 kD, more preferably from about 10 kD to about 35 kD. The molecular weight range of PLA is from about 20 to about 30 kDa. The ratio of lactide to glycolide is from about 75:25 to about 50:50. In one embodiment, the ratio is 50:50.

Exemplary polymers include, but are not limited to, poly(D,L-lactic-co-glycolic acid) (PLGA, 50:50 lactic acid to glycolic acid ratio, Mₙ=10 kDa, referred to as 502H); poly(D,L-lactic-co-glycolic acid) (PLGA, 50:50 lactic acid to glycolic acid ratio, Mₙ=25 kDa, referred to as 503H); poly(D,L-lactic-co-glycolic acid) (PLGA, 50:50 lactic acid to glycolic acid ratio, Mₙ=30 kDa, referred to as 504H); poly(D,L-lactic-co-glycolic acid) (PLGA, 50:50 lactic acid to glycolic acid ratio, Mₙ=35 kDa, referred to as 504); and poly(D,L-lactic-co-glycolic acid) (PLGA, 75:25 lactic acid to glycolic acid ratio, Mₙ=10 kDa, referred to as 752).

The microsphere compositions described herein can release an effective amount of one or more active agents, for example, agents suitable for managing elevated IOP, for a period greater than 14 days in vivo, preferably greater than 60 days in vivo, more preferably up to 73 days in vivo, more preferably greater than 90 days in vivo, even more preferably over 100 days in vivo, and most preferably greater than 107 days in vivo. Release for a period of 90 days or greater corresponds to the typical time period between ophthalmologist visits for patients suffering from glaucoma. The sustained release of drug, in combination with the ability to administer the drug in a minimally invasive manner, should increase patient compliance.

In other embodiments, the drug is released over a period of greater than 150 days, more preferably over 200 days, more preferably over 250 days, most preferably up to 270 days or longer.

The examples show that release is influenced by a variety of factors, including molecular weight of the polymer, hydrophilicity or hydrophobicity of the polymer, percent loading of the drug, and/or methods of manufacturing the microspheres. For example, release of prednisolone acetate was less at a given time period for microspheres prepared from PLGA 50211 compared to microspheres prepared from PLGA 503H and a blend of PLGA 50211 and PLA. Release of prednisolone acetate is also influenced by the method in which the microspheres are prepared. PLGA 50211 microspheres prepared using sonication or homogenization exhibited greater release than PLGA 502H microspheres prepared without sonication or homogenization or prepared with PEG 1500.

With respect to AG1478, release was greater for PLGA 503H microspheres prepared using an oil-in-water emulsion technique having a drug loading of 5.0% compared to a loading of 2.5%. The microspheres exhibited a more rapid release of drug over the first 50 days, followed by a more linear release over the next 125 days.

Hydrophilicity of the polymer influences the release profile of AG 1478. For example, release of AG1478 was greater from microspheres prepared from PLGA having carboxylic end groups, such as PLGA 503H and 504H, compared to the non-carboxylated polymer, PLGA 504, using the oil-in-water cosolvent technique. This is likely due to the fact that AG1478 interacts more strongly with the less hydrophilic polymer PLGA 504, thus slowing the rate of release.

In one embodiment, the microparticles are formed from poly(D,L-lactic-co-glycolic acid (PLGA, 50:50 lactic acid to glycolic acid ratio, Mₙ=10 kDa, referred to as 502H). In another embodiment, the microparticles are formed from poly(D,L-lactic-co-glycolic acid (PLGA, 50:50 lactic acid to glycolic acid ratio, Mₙ=25 kDa, referred to as 503H). In still another embodiment, the microparticles are formed from poly(D,L-lactic-co-glycolic acid (PLGA, 50:50 lactic acid to glycolic acid ratio, Mₙ=30 kDa, referred to as 504H). In yet another embodiment, the microparticles are formed from poly(D,L-lactic-co-glycolic acid (PLGA, 50:50 lactic acid to glycolic acid ratio, Mₙ=35 kDa, referred to as 504). In still another embodiment, the microparticles are formed from poly(D,L-lactic-co-glycolic acid (PLGA, 75:25 lactic acid to glycolic acid ratio, Mₙ=10 kDa, referred to as 752). In yet another embodiment, the microparticles are prepared from a blend of PLGA and PL (referred to as PLGA:PL). The designation “H” means the polymer is terminated with a carboxylic acid group. Microparticles can also be prepared from polylactic acid. As demonstrated by the examples, the H polymers are preferred for loading of hydrophilic drug.

C. Solvents and Surfactants for Preparation of Microparticles

Typical solvents are organic solvents such as methylene chloride, which leave low levels of residue that are generally accepted as safe. Suitable water-insoluble solvents include methylene chloride, chloroform, carbon tetrachloride, dichlorethane, ethyl acetate and cyclohexane. Additional solvents include, but are not limited to, alcohols such as methanol (methyl alcohol), ethanol, (ethyl alcohol), 1-propanol (n-propyl alcohol), 2-propanol (isopropyl alcohol), 1-butanol (n-butyl alcohol), 2-butanol (sec-butyl alcohol), 2-methyl-1-propanol (isobutyl alcohol), 2-methyl-2-propanol (t-butyl alcohol), 1-pentanol (n-pentyl alcohol), 3-methyl-1-butanol (isopentyl alcohol), 2,2-dimethyl-1-propanol (neopentyl alcohol), cyclopentanol (cyclopentyl alcohol), 1-hexanol (n-hexanol), cyclohexanol (cyclohexyl alcohol), 1-heptanol (n-heptyl alcohol), 1-octanol (n-octyl alcohol), 1-nonanol (n-nonyl alcohol), 1-decanol (n-decyl alcohol), 2-propen-1-ol (allyl alcohol), phenylmethanol (benzyl alcohol), diphenylmethanol (diphenylcarbinol), triphenylmethanol (triphenylcarbinol), glycerin, phenol, 2-methoxyethanol, 2-ethoxyethanol, 3-ethoxy-1,2-propanediol, Di(ethylene glycol) methyl ether, 1,2-propanediol, 1,3-propanediol, 1,3-butanediol, 2,3-butanediol, 1,4-butanediol, 1,2-pentanediol, 1,3-pentanediol, 1,4-pentanediol, 1,5-pentanediol, 2,3-pentanediol, 2,4-pentanediol, 2,5-pentanediol, 3,4-pentanediol, 3,5-pentanediol, and combinations thereof.

D. Excipients for Administration to the Eye

Considerations in the formulation of the microsphere compositions include, but are not limited to, sterility, preservation, isotonicity, and buffering. The preparation of ophthalmic solutions and suspensions are described in Ansel et al., Pharmaceutical Dosage Forms and Drug Delivery Systems 6^{th} Ed., pp. 396-408, Williams and Wilkins (1995). Suspensions are often more advantageous than solutions as they typically have increased corneal contact time and thus can provide higher efficacy. Ophthalmic suspensions must contain particles of appropriate chemical characteristics and size to be non-irritating to the eyes. The suspension must also not agglomerate upon administration. Excipients, such as dispersants, can be included to prevent aggregation of the particles.

The microspheres are typically suspended in sterile saline, phosphate buffered saline, or other pharmaceutically acceptable carriers for administration to the eye.

Materials that may be used to formulate or prepare the microparticles include anionic, cationic, amphoteric, and non-ionic surfactants. Anionic surfactants include di-(2 ethylhexyl) sodium sulfosuccinate; non-ionic surfactants include the fatty acids and the esters thereof; surfactants in the amphoteric group include (1) substances classified as simple, conjugated and derived proteins such as the albumins, gelatins, and glycoproteins, and (2) substances contained within the phospholipid classification, for example, lecithin. The amine salts and the quaternary ammonium salts within the cationic group also comprise useful surfactants. Other surfactant compounds useful to form coacervates include polysaccharides and their derivatives, the mucopolysaccharides and the polysorbates and their derivatives. Synthetic polymers that may be used as surfactants include compositions such as polyethylene glycol and polypropylene glycol. Further examples of suitable compounds that may be utilized to prepare coacervate systems include glycoproteins, glycolipids, galactose, gelatins, modified fluid gelatins and galacturonic acid. In one embodiment, the surfactant is polyvinyl alcohol.

Hydrophobic surfactants such as fatty acids and cholesterol are added during processes to improve the resulting distribution of hydrophobic drugs in hydrophobic polymeric microparticles. Examples of fatty acids include butyric acid, valeric acid, caproic acid, enanthic acid, caprylic acid, pelargonic acid, caprylic acid, undecylic acid, lauric acid, tridecylic acid, myristic acid, pentadecylic acid, palmitic acid, heptadecylic acid, stearic acid, nonadecanoic acid, arachic acid, isocrotonic acid, undecylenic acid, oleic acid, elaidic acid, sorbic acid, linoleic acid, linolenic acid and arachidonic acid.

In those embodiments where hydrophilic drugs are encapsulated, hydrophilic surfactant can be added during particle manufacture to improve the resulting distribution of hydrophilic drugs in hydrophilic polymeric microparticles. Hydrophilic surfactants generally have an HLB higher than 10, for example, 16-18. The hydrophilic surfactant can be ionic or nonionic. Examples of hydrophilic surfactants are known in the art and include phospholipids, polyoxyethylene sorbitan fatty acid derivatives, castor oil or hydrogenated castor oil ethoxylates, fatty acid ethoxylates, alcohol ethoxylates, polyoxyethylene, polyoxypropylene co-polymers and block co-polymers; anionic surfactants, and alkylphenol surfactants.

### III. Methods of Making

There are several processes whereby microparticles can be made, including, but not limited to, spray drying, interfacial polymerization, hot melt encapsulation, phase separation encapsulation, spontaneous emulsion, solvent evaporation microencapsulation, solvent removal microencapsulation, coacervation, low temperature microsphere formation, and phase inversion nanoencapsulation (“PIN”).

The dispersion of the one or more active agents within the polymer matrix can be enhanced by varying: (1) the solvent used to solvate the polymer; (2) the ratio of the polymer to the solvent; (3) the particle size of the material to be encapsulated; (4) the percentage of the active agent(s) relative to the polymer (e.g., drug loading); and/or the polymer concentration.

The following are representative methods for forming microparticles.

Spray Drying

In spray drying, the core material to be encapsulated is dispersed or dissolved in a solution. Typically, the solution is aqueous and preferably the solution includes a polymer. The solution or dispersion is pumped through a micronizing nozzle driven by a flow of compressed gas, and the resulting aerosol is suspended in a heated cyclone of air, allowing the solvent to evaporate from the microdroplets. The solidified microparticles pass into a second chamber and are trapped in a collection flask.

Interfacial Polycondensation

Interfacial polycondensation is used to microencapsulate a core material in the following manner. One monomer and the core material are dissolved in a solvent. A second monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

Hot Melt Encapsulation

In hot melt microencapsulation, the core material (to be encapsulated) is added to molten polymer. This mixture is suspended as molten droplets in a nonsolvent for the polymer (often oil-based) which has been heated to approximately 10° C. above the melting point of the polymer. The emulsion is maintained through vigorous stirring while the nonsolvent bath is quickly cooled below the glass transition of the polymer, causing the molten droplets to solidify and entrap the core material.

Solvent Evaporation Microencapsulation

In solvent evaporation microencapsulation, the polymer is typically dissolved in a water immiscible organic solvent and the material to be encapsulated is added to the polymer solution as a suspension or solution in an organic solvent. An emulsion is formed by adding this suspension or solution to a beaker of vigorously stirring water (often containing a surface active agent, for example, polyethylene glycol or polyvinyl alcohol, to stabilize the emulsion). The organic solvent is evaporated while continuing to stir. Evaporation results in precipitation of the polymer, forming solid microcapsules containing core material.

The solvent evaporation process can be used to entrap a liquid core material in a polymer such as PLA, PLA/PGA copolymer, or PLA/PCL copolymer microcapsules. The polymer or copolymer is dissolved in a miscible mixture of solvent and nonsolvent, at a nonsolvent concentration which is immediately below the concentration which would produce phase separation (i.e., cloud point). The liquid core material is added to the solution while agitating to form an emulsion and disperse the material as droplets. Solvent and nonsolvent are vaporized, with the solvent being vaporized at a faster rate, causing the polymer or copolymer to phase separate and migrate towards the surface of the core material droplets. This phase-separated solution is then transferred into an agitated volume of nonsolvent, causing any remaining dissolved polymer or copolymer to precipitate and extracting any residual solvent from the formed membrane. The result is a microcapsule composed of polymer or copolymer shell with a core of liquid material.

Solvent evaporation microencapsulation can result in the stabilization of insoluble or poorly soluble drug particles in a polymeric solution for a period of time ranging from 0.5 hours to several months.

The stabilization of insoluble or poorly soluble drug particles within the polymeric solution could be critical during scale-up. By stabilizing suspended drug particles within the dispersed phase, said particles can remain homogeneously dispersed throughout the polymeric solution as well as the resulting polymer matrix that forms during the process of microencapsulation. The homogeneous distribution of drug particles can be achieved in any kind of device, including microparticles, nanoparticles, rods, films, and other device.

Solvent evaporation microencapsulation (SEM) has several advantages. SEM allows for the determination of the best polymer-solvent-insoluble particle mixture that will aid in the formation of a homogeneous suspension that can be used to encapsulate the particles. SEM stabilizes the insoluble particles or within the polymeric solution, which will help during scale-up because one will be able to let suspensions of insoluble particles sit for long periods of time, making the process less time-dependent and less labor intensive. SEM allows for the encapsulated particles to remain suspended within a polymeric solution for up to 30 days, which may increase the amount of insoluble material entrapped within the polymeric matrix, potentially improving the physical properties of the drug delivery vehicle. SEM allows for the creation of microparticles or nanoparticles that have a more optimized release of the encapsulated material. For example, if the insoluble particle is localized to the surface of the microparticle or nanoparticle, the system will have a large ‘burst’ effect. In contrast, creating a homogeneous dispersion of the insoluble particle within the polymeric matrix will help to create a system with release kinetics that begin to approach the classical ‘zero-ordered’ release kinetics that are often perceived as being ideal in the field of drug delivery).

In a preferred embodiment, the microspheres are prepared using an oil-in-water emulsion cosolvent technique, in which an organic cosolvent, such as DMSO, is used to prepare the microspheres.

Solvent Removal Microencapsulation

In solvent removal microencapsulation, the polymer is typically dissolved in an oil miscible organic solvent and the material to be encapsulated is added to the polymer solution as a suspension or solution in organic solvent. Surface active agents can be added to improve the dispersion of the material to be encapsulated. An emulsion is formed by adding this suspension or solution to vigorously stirring oil, in which the oil is a nonsolvent for the polymer and the polymer/solvent solution is immiscible in the oil. The organic solvent is removed by diffusion into the oil phase while continuing to stir. Solvent removal results in precipitation of the polymer, forming solid microcapsules containing core material.

Phase Separation Microencapsulation

In phase separation microencapsulation, the material to be encapsulated is dispersed in a polymer solution with stirring. While continually stirring to uniformly suspend the material, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the core material in a droplet with an outer polymer shell.

Spontaneous Emulsification

Spontaneous emulsification involves solidifying emulsified liquid polymer droplets by changing temperature, evaporating solvent, or adding chemical cross-linking agents. The physical and chemical properties of the encapsulant, and the material to be encapsulated, dictates the suitable methods of encapsulation. Factors such as hydrophobicity, molecular weight, chemical stability, and thermal stability affect encapsulation.

Coacervation

Encapsulation procedures for various substances using coacervation techniques have been described in the prior art, for example, in GB-B-929 406; GB-B-929 401; U.S. Pat. Nos. 3,266,987; 4,794,000 and 4,460,563. Coacervation is a process involving separation of colloidal solutions into two or more immiscible liquid layers (Ref. Dowben, R. General Physiology, Harper & Row, New York, 1969, pp. 142-143.). Through the process of coacervation compositions comprised of two or more phases and known as coacervates may be produced. The ingredients that comprise the two phase coacervate system are present in both phases; however, the colloid rich phase has a greater concentration of the components than the colloid poor phase.

Phase Inversion Nanoencapsulation (“PIN”)

A preferred process is PIN. In PIN, a polymer is dissolved in an effective amount of a solvent. The agent to be encapsulated is also dissolved or dispersed in the effective amount of the solvent. The polymer, the agent and the solvent together form a mixture having a continuous phase, wherein the solvent is the continuous phase. The mixture is introduced into an effective amount of a nonsolvent to cause the spontaneous formation of the microencapsulated product, wherein the solvent and the nonsolvent are miscible. PIN has been described by Mathiowitz et al. in U.S. Pat. Nos. 6,131,211 and 6,235,224. A hydrophobic agent is dissolved in an effective amount of a first solvent that is free of polymer. The hydrophobic agent and the solvent form a mixture having a continuous phase. A second solvent and then an aqueous solution are introduced into the mixture. The introduction of the aqueous solution causes precipitation of the hydrophobic agent and produces a composition of micronized hydrophobic agent having an average particle size of 1 micron or less.

An improved process is demonstrated in the examples. The process uses a mixed solvent including at least one water-insoluble solvent and water that contains a surfactant, such as PVA. The drug is either dissolved or dispersed together with a substance that has a high molecular weight (such as a polymer) into an organic solvent composition, optionally containing non-ionic surfactants of various hydrophilic-lipophilic ratios. The composition is then introduced into an aqueous solution that contains a surfactant like PVA. The water-insoluble solvent forms an oil phase (inner phase) and is stirred into the aqueous solution as a water phase (outer phase). The O/W emulsion is combined with fresh water that contains surfactant such as PVA and is stirred to help aid the solvent evaporation. The aqueous solution contains an activator such as polyvinyl alcohol, whereby the oil phase is enclosed as small droplets within the aqueous solution as shells. The proportion of the water-miscible solvent in the oil phase is from 5% to 95%. An important aspect of this improved method is the use of high shear during the initial mixing phase, which is achievable, for example, using sonication for a period of one hour, with stirring, to uniformly mix in high amounts of drug particles in the polymer liquefied by dissolution or by melting.

Melt-Solvent Evaporation Method

In the melt-solvent evaporation method, the polymer is heated to a point of sufficient fluidity to allow ease of manipulation (for example, stirring with a spatula). The temperature required to do this is dependent on the intrinsic properties of the polymer. For example, for crystalline polymers, the temperature will be above the melting point of the polymer. After reaching the desired temperature, the agent is added to the molten polymer and physically mixed while maintaining the temperature. The molten polymer and agent are mixed until the mixture reaches the maximum level of homogeneity for that particular system. The mixture is allowed to cool to room temperature and harden. This may result in melting of the agent in the polymer and/or dispersion of the agent in the polymer. This can result in an increase in solubility of the drug when the mixture is dissolved in organic solvent. The process is easy to scale up since it occurs prior to encapsulation. High shear turbines may be used to stir the dispersion, complemented by gradual addition of the agent into the polymer solution until the desired high loading is achieved. Alternatively the density of the polymer solution may be adjusted to prevent agent settling during stirring.

This method increases microparticle loading as well as uniformity of the resulting microparticles and of the agent within the microparticles. When an agent is formed into microspheres by double-emulsion solvent evaporation, transfer of the agent from the inner phase to the outer water phase can be prevented. This makes it possible to increase the percentage of agent entrapped within the microspheres, resulting in an increased amount of the drug in the microspheres.

The distribution of the agent in particles can also be made more uniform. This can improve the release kinetics of the agent. Generally, the agent is dissolved or dispersed together with a substance that has a high molecular weight in an organic solvent composition; with or without non-ionic surfactants of various hydrophilic-lipophilic ratios. The composition is introduced into an aqueous solution that contains a surfactant like PVA. The water-insoluble solvent forms an oil phase (inner phase) and is stirred into the aqueous solution as a water phase (outer phase). The O/W emulsion is combined with fresh water that contains PVA and is stirred to help aid the solvent evaporation. The aqueous solution contains an activator such as polyvinyl alcohol, whereby the oil phase is enclosed as small droplets within the aqueous solution as shells.

In one embodiment, the microparticles are formed using a water-in-oil double emulsion (w/o/w) solvent evaporation technique. For example, the one or more active agents are dissolved in deionized water. The polymer is dissolved in an organic solvent or cosolvent. The aqueous and organic phases are emulsified via vortexing to obtain the desired active agent to polymer ratio (e.g., 10%, 20%, or greater). The emulsion is then added dropwise to an aqueous solution of a surfactant (such as polyvinyl alcohol) and allowed to stir/harden for 3 hours. The resulting microparticles are collected, such as by centrifugation, washed with deionized water, and dried (e.g., freeze drying).

A comparison of the release profiles of the three emulsion methods used in the examples to prepare AG1478 microspheres revealed a direct relationship between the encapsulation method and the amount released. As the solubility of the drug increased, i.e. from the s/o/w emulsion to the o/w emulsion, the amount of drug encapsulated (from 10.8 μg/mg to 13.5 μg/mg) and released increased.

The other effect observed involved the significant increase in the initial burst after 1 day of release in the o/w co-solvent compared to the other emulsions. This increase in burst is likely a result of the significant reduction in the size of the microspheres. Smaller sphere size increases the number of spheres per unit volume and thus increases the surface area, leading to more drug released in a shorter period. While the o/w co-solvent exhibited an increased burst, spheres prepared by this method sustained the longest release of AG1478—over 9 months (266 days). Furthermore, the amount of drug released at later time points in all fabrication methods were on average greater than 3 mM (˜9.5 ng/mg polymer), an amount more than sufficient to achieve greater than 95% inhibition in the FR3T3 and A431 cells, as well as other cancer cell types.

As demonstrated by the examples, the percent loading is increased by “matching” the hydrophilicity or hydrophobicity of the polymer to the agent to be encapsulated. In some cases, such as PLGA, this can be achieved by selecting the monomer ratios so that the copolymer is more hydrophilic. Alternatively, the polymer can be made more hydrophilic, for example, by treating the polymer with a carboxyl solution. A combination of a hydrophilic drug and a hydrophobic drug can be encapsulated in microparticles prepared from a blend of a more hydrophilic PLGA and a hydrophobic polymer, such as PLA.

### IV. Methods of Use

A. Disorders to be Treated

The microsphere compositions described herein can be administered to treat or prevent diseases or disorders, most preferably of the eye. The dosage of the drug which is released at the site of administration should be bioequivalent as defined by the Food and Drug Administration for the drug when administered in solution, suspension or enterally, in the absence of the microparticles.

Glaucoma

In one embodiment, the microsphere compositions can be administered to manage (e.g., reduce) IOP in patients needing such treatment, for example, patients suffering from glaucoma. Glaucoma is an ophthalmic disease characterized by the gradual degeneration of retinal ganglion cells (RGCs). RGCs synapse with bipolar cells and transmit visual inputs to the brain along the optic nerve. Degeneration of these cells leads to gradual vision loss and ultimately blindness if untreated.

The microspheres described herein can also be used to deliver one or more active agents that promote neural regeneration, for example, in patients suffering from glaucoma. AG1478 has been shown to promote neural regeneration. AG1478 is an inhibitor of EGFR.

The neural degeneration in glaucoma is accompanied by extensive remodeling of the extracellular matrix (ECM) including the production of chondroitin sulfate proteoglycans (CSPGs) which inhibit regeneration. Administration of an EGFR inhibitor, such as AG1478, has been shown to lead to a reduction in activated astrocytes, a reduction in the production of CSPGs, and regeneration in the optic nerve.

AG1478 can be co-administered with neural progenitor cells to replace lost retinal ganglion cells (RGCs) along with sustained delivery of AG1478 to promote regeneration. The optic nerve crush model is an excellent first model for studying methods to promote regeneration in glaucoma as well as in the CNS more broadly.

Recent work suggests that the EGFR plays an important role in regulating the production of CSPGs and maintaining specific astrocyte phenotype. EGFR, also known as human EGF receptor (HER) and ErbB1, is a member of a family of transmembrane proteins with tyrosine kinase activity. EGFR has seven different but structurally similar ligands, including EGF, transforming growth factor-β1 (TGF-PI), and transforming growth factor-α (TGF-α). EGFR activation controls cell migration, apoptosis, protein secretion and differentiation. Activation of EGFR has been shown to affect the behavior of astrocytes. Ligands of EGFR stimulate astrocyte proliferation and differentiation, induce morphological changes and process formation, and enhance their mobility in vitro. In glaucomatous optic neuropathy, EGFR activation is increased in astrocytes and their activation in the cribriform plates to the damaged optic nerve bundles creates compression, backward bowing, and disorganization of the optic nerve head-characteristic features of glaucomatous eyes with high or normal intraocular pressure.

The EGFR ligands EGF and TGF-β1 greatly increase CSPG production after injury, including neurocan and phosphacan, while upregulation of CSPGs by astrocytes is mediated specifically by the EGFR receptor. In addition, activation of EGFR causes optic nerve astrocytes and brain astrocytes to form cribriform structures with cavernous spaces, similar to the structures that reactive astrocytes form in the glial scar. EGFR also plays a role in astrocyte phenotype. In normal tissue astrocytes are quiescent, producing only a moderate amount of CSPGs and retaining a stellate morphology. After injury, these quiescent astrocytes are activated and become reactive, with elongated processes and increased motility. Astrocytes upregulate and activate EGFR in three different optic nerve injury models: transient eye ischemia, chronic glaucoma, and optic nerve transection. However, application of a commercially available EGFR tyrosine kinase inhibitor, AG1478-a potent, reversible antagonist of EGFR-in a rodent model of glaucomatous optic neuropathy and an optic nerve crush model, reverses this upregulation and activation of astrocytes and increases the survival of RGCs. Further, evidence shows that EGFR activation mediates inhibition of axon regeneration in retinal explants by production of CSPGs and myelin. These studies provide evidence for the idea that modulating the behavior of astrocytes via EGFR signaling is an attractive candidate for treatment of CNS disorders.

Uveitis

Uveitis specifically refers to inflammation of the middle layer of the eye, termed the “uvea” but in common usage may refer to any inflammatory process involving the interior of the eye. Uveitis is estimated to be responsible for approximately 10% of the blindness in the United States. Uveitis requires an urgent referral and thorough examination by an ophthalmologist, along with urgent treatment to control the inflammation.

Uveitis is usually categorized anatomically into anterior, intermediate, posterior and panuveitic forms. Anywhere from two-thirds to 90% of uveitis cases are anterior in location (anterior uveitis), frequently termed iritis—or inflammation of the iris and anterior chamber. This condition can occur as a single episode and subside with proper treatment or may take on a recurrent or chronic nature. Symptoms include red eye, injected conjunctiva, pain and decreased vision. Signs include dilated ciliary vessels, presence of cells and flare in the anterior chamber, and keratic precipitates (“KP”) on the posterior surface of the cornea. Intermediate uveitis consists of vitritis—inflammatory cells in the vitreous cavity, sometimes with snowbanking, or deposition of inflammatory material on the pars plana. Posterior uveitis is the inflammation of the retina and choroid. Pan-uveitis is the inflammation of all the layers of the uvea.

Myriad conditions can lead to the development of uveitis, including systemic diseases as well as syndromes confined to the eye. In anterior uveitis, no specific diagnosis is made in approximately one-half of cases. However, anterior uveitis is often one of the syndromes associated with HLA-B27.

The prognosis is generally good for those who receive prompt diagnosis and treatment, but serious complication (including cataracts, glaucoma, band keratopathy, retinal edema and permanent vision loss) may result if left untreated. The type of uveitis, as well as its severity, duration, and responsiveness to treatment or any associated illnesses, all factor in to the long term prognosis. Uveitis can be treated using steroids, such as prednisolone, and chemotherapeutic agents, such as methotrexate. In a preferred embodiment, the microspheres are loaded with ofloxacin, prednisolone, or a combination thereof. The microspheres preferably provide release of the one or more active agents for a period of between 14 and 21 days. In another embodiment, the microspheres provide sustained release of the one or more active agents over a period greater than three weeks, preferably over a period of greater than 49 days, more preferably over a period of two months, most preferably over a period of three months.

Post Surgical Ocular Inflammation/Infection

Most surgeries involving the eye are followed by ocular inflammation and/or infection. Topical administration of eye drops containing a combination of a steroid and an antibiotic is the predominant treatment for controlling inflammation as well as infection. Although such eye drops have been shown to be effective, poor compliance and the risk of re-opening of the stitched wound due to continuous touching of the wound when applying the eye drops remain fundamental issues. Therefore, it is desirable to provide a long-term ocular delivery system that provides release of the active agents for approximately 2-3 weeks in order to minimize dosing frequency, improve patient compliance, reduce side effects due to systemic absorption of the active agents, and keep the stitched wound intact. In one embodiment, microspheres loaded with an antibiotic, a steroid, or combinations thereof are administered to a patient post eye surgery. In a preferred embodiment, the microspheres are loaded with ofloxacin, prednisolone, or a combination thereof. The microspheres preferably provide release of the one or more active agents for a period of between 14 and 21 days. In another embodiment, the microspheres provide sustained release of the one or more active agents over a period greater than three weeks, preferably over a period of greater than 49 days, more preferably over a period of two months, most preferably over a period of three months.

Dry Eye Syndrome

Dry eye syndrome (Keratoconjunctivitis sicca (KCS)) is one of the most common problems treated by eye physicians. Over ten million Americans suffer from dry eyes. It is usually caused by a problem with the quality of the tear film that lubricates the eyes.

Dry eye syndrome has many causes. One of the most common reasons for dryness is simply the normal aging process. As we grow older, bodies produce less oil—60% less at age 65 then at age 18. This is more pronounced in women, who tend to have drier skin then men. The oil deficiency also affects the tear film. Without as much oil to seal the watery layer, the tear film evaporates much faster, leaving dry areas on the cornea.

Many other factors, such as hot, dry or windy climates, high altitudes, air-conditioning and cigarette smoke also cause dry eyes. Contact lens wearers may also suffer from dryness because the contacts absorb the tear film, causing proteins to form on the surface of the lens. Certain medications, thyroid conditions, vitamin A deficiency, and diseases such as Parkinson's and Sjogren's can also cause dryness.

Inflammation occurring in response to tears film hypertonicity can be treated by administering the microspheres described herein loaded with poorly water soluble steroids and/or with poorly water soluble immunosuppressants.

Macular Degeneration

Macular degeneration is a medical condition predominantly found in elderly adults in which the center of the inner lining of the eye, known as the macula area of the retina, suffers thinning, atrophy, and in some cases, bleeding. This can result in loss of central vision, which entails inability to see fine details, to read, or to recognize faces. According to the American Academy of Ophthalmology, it is the leading cause of central vision loss (blindness) in the United States today for those over the age of fifty years. Although some macular dystrophies that affect younger individuals are sometimes referred to as macular degeneration, the term generally refers to age-related macular degeneration (AMD or ARMD). Macular degeneration can be treated using anti-angiogenesis inhibitors. In one embodiment, the microspheres are loaded with a poorly water soluble anti-angiogenesis inhibitor or growth factor for the treatment of macular degeneration.

B. Methods of Administration

The composition can be administered using a variety of techniques well known in the art including, but not limited to, topically and by injection. Suitable dosage forms include but are not limited to, ointments and solutions and suspensions, such as eye drops. In one embodiment, the compositions are administered to the eye by injection. In a preferred embodiment, the microsphere composition is administered subconjunctivally. “Subconjunctival” or “subconjunctivally”, as used herein, refers to administration under the conjunctiva of the eye. The conjunctiva is the clear membrane that coats the inner aspect of the eyelids and the outer surface of the eye. The microsphere compositions are generally administered as suspensions in a pharmaceutically acceptable carrier, such as phosphate buffered saline (PBS). Subconjunctival administration of drugs, typically by injection, has shown minimal concentration of drug in the plasma and notable concentrations in the eye, including the aqueous humor.

### V. Kits

The kits contain the microsphere compositions and optionally one or more pharmaceutically acceptable excipients or carriers. In one embodiment, the kit can contain the microspheres in dry powder form in one container, such as a vial, jar, or ampule, and the pharmaceutically acceptable carrier in another container, such as a vial, jar, or ampule. The kit typically contains instructions for resuspending the microparticles in the carrier and for administering the composition. If excipients are present, they can be in one or both containers.

In another embodiment, the kit can contain the microparticles resuspended in the carrier and optionally one or more pharmaceutically excipients. The kit would typically contain instructions for administering the composition. The kit can also contain one or more apparatus for preparing and/or administering the compositions, such as a needle and syringe. The container(s) containing the microspheres and the carrier can be packaged using techniques well known in the art. Suitable package materials include, but are not limited to, boxes

Unless defined otherwise, all technical and scientific terms used herein have the same meanings as commonly understood by one of skill in the art to which the disclosed invention belongs.

### EXAMPLES

### Materials

poly(D,L-lactic-co-glycolic acid (PLGA, 50:50 lactic acid to glycolic acid ratio, Mₙ=10 kDa, referred to as 50211); poly(D,L-lactic-co-glycolic acid (PLGA, 50:50 lactic acid to glycolic acid ratio, Mₙ=25 kDa, referred to as 503H); poly(D,L-lactic-co-glycolic acid (PLGA, 50:50 lactic acid to glycolic acid ratio, Mₙ=30 kDa, referred to as 504H); and poly(D,L-lactic-co-glycolic acid (PLGA, 50:50 lactic acid to glycolic acid ratio, Mₙ=35 kDa, referred to as 504) were purchased from Boehringer Ingelheim (Ingelheim, Germany). The designation “H” means the polymer is terminated with a carboxylic acid group.

Poly(D,L-lactic acid) (PLA, Mₙ=˜20-30 kDa) and poly(vinyl alcohol) (PVA, 88 mol % hydrolyzed) were purchased from Polyscienes (Warrington, Pa., USA).

Methotrexate and Prednisolone acetate were purchased from Sigma (St. Louis, Mo., USA).

All other chemicals were A.C.S. reagent grade from Sigma (St. Louis, Mo., USA).

### Methods

Plotting UV absorbance versus drug concentration produced a calibration curve for quantification of drug. For release time and loading, a linear fit was produced for each drug. The resulting curves were used to determine loading and release characteristics for the microspheres.

To determine loading of drug in the microspheres, an amount of drug-loaded microspheres or blank microspheres were placed in 1.5 mL tubes. The microspheres were dissolved in solvent, such as DMSO. The concentration was determined using spectrophotometry. Encapsulation efficiency was determined by dividing the actual amount of drug loaded into the microspheres by the amount of drug added to the emulsion preparation.

In 1.5 mL tubes, an amount of microspheres or blank microspheres were reconstituted in a solvent, typically 1 mL of phosphate buffered saline (PBS, pH 7,4), Samples were typically prepared in triplicate. Mixtures were then incubated at 37° C. on a rotating shaker (e.g., Barnstead/Thermolyne; Dubuque, Iowa). At specific time points—1, 5 and 8 h and 1, 3, 5 and 7 days and once every 7 days afterward until no drug could be measured—the mixture was centrifuged and the supernatant collected. One milliliter of PBS was added to the tubes to replace the supernatant and the mixture was then vortexed to resuspend the microspheres. The tubes were returned to the shaker until the next time point. Collected supernatants were stored at −20° C. until they could be analyzed by UV spectroscopy.

The volume-weighted mean diameter of the microspheres from each batch was measured using a Beckman Coulter Multisizer 3 (Fullerton, Calif.) with a 50 mm aperture and a sample size of at least 5000 microspheres. Morphology was examined with scanning electron microscopy (SEM). Samples were mounted and sputter coated with gold for 30 s at 40 mA. Micrographs were taken on a Philips XL-30 environmental SEM operating at 10 kV.

### Example 1

### Preparation of Microspheres

The microspheres were prepared by phase separation using a single emulsion solvent evaporation method. Two hundred milligrams of the specific polymer was dissolved in 1 mL of dichloromethane (DCM) and 4 mL of trifluoroethanol (TFE). 40 mg of prednisolone acetate or 20 mg of methotrexate was added to the polymer solution and vortexed to obtain the desired drug to polymer ratio: 20% (40 mg drug/200 mg polymer) for prednisolone acetate and 10% (20 mg drug/200 mg polymer) for methotrexate. The organic phase was added dropwise to 200 mL of 5% (w/v) PVA aqueous solution. The aqueous and organic phases were emulsified via stirring/hardening for three hours. Microspheres were collected by centrifugation, washed three times with deionized water, and freeze dried for three days. Blank microspheres were made at the same time under identical conditions except no drug was added.

### Particle Sizing and Scanning Electron Microscopy

The volume-weighted mean diameter of the microparticles from each batch can be determined using a Beckman Coulter Multisizer 3 (Fullerton, Calif., USA) with a 100 μm diameter aperture based on a sample size of at least 80,000 microspheres. Scanning electron microscopy (SEM) analysis can be used to examine the morphology of the spheres. Microspheres can be sputter coated with gold for 30 seconds at 25 mA and SEM micrographs can be taken on a FEI XL-30 environmental SEM operating at 4 kV.

### Example 2

### In Vitro Release Studies of Prednisolone Acetate-Loaded Microspheres

In 1.5 mL eppendorf tubes, 10 mg prednisolone acetate-loaded microspheres or blank microspheres were suspended in 1 mL of phosphate buffered saline (PBS). Samples were prepared in triplicate. The mixtures were incubated at 37° C. on a labquake rotating shaker. At specific time points, 1, 3, 5, and 8 hours and 1, 3, and 7 days, and once every 7 days thereafter until no pellets were present, the mixture was centrifuged and the supernatant and the supernatant was collected. One milliliter of phosphate buffered saline was then added to replace the withdrawn supernatant and the microspheres were resuspended and returned to the shaker. Supernatants for each of the sets of microspheres was frozen and stored at −80° C. for subsequent analysis using UV spectroscopy at 245 nm and 303 nm for prednisolone acetate and methotrexate respectively. The concentration of dissolved prednisolone acetate or methotrexate was determined as a function of from their respective standard curves. Plotting prednisolone acetate or methotrexate concentration versus UV absorbance produced a calibration curve for quantification of prednisolone acetate or methotrexate. A linear fit was established from −0.09-25 μg/ml, of prednisolone acetate (Y=26.798x+0.185 1; r²=0.9997) or methotrexate (Y=19.763x+0.0372; r²=1) in phosphate buffered saline.

In Vitro Release of Prednisolone Acetate

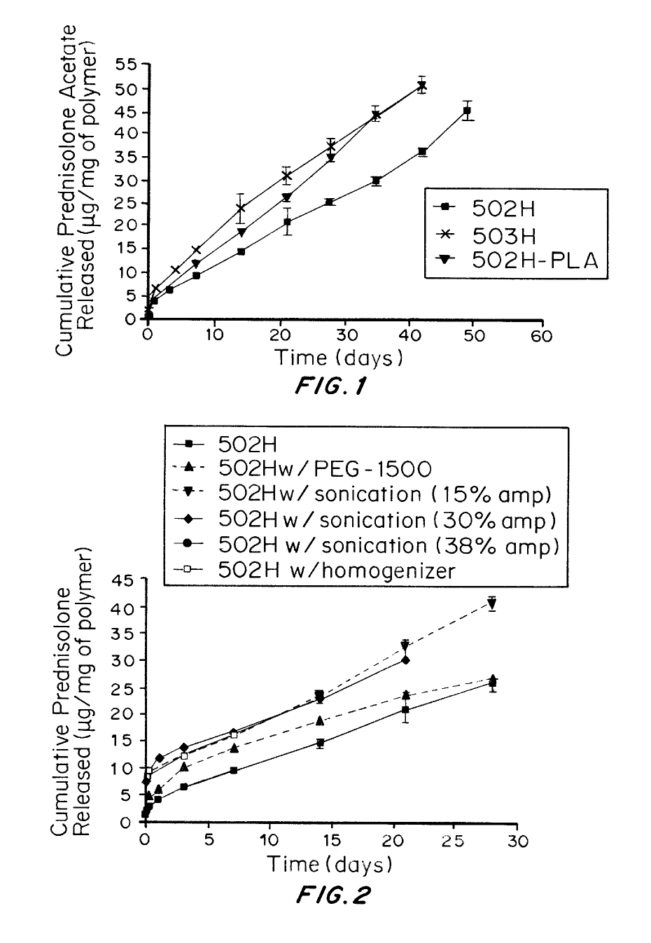

FIG. 1 shows the in vitro release profile for 20% prednisolone acetate-loaded microspheres. Prednisolone acetate was released less rapidly from PLGA 502H microspheres than from microspheres prepared from PLGA 503H or a blend of PLGA 502H and PLA.

Such long sustained release of prednisolone acetate can cause adverse side effects. Therefore the techniques for preparing the microparticles were modified in an attempt to reduce the period of release to 14-21 days. Three different modifications were made during preparation of the microspheres:

(a) Addition of 20 mg of PEG 1500 to the solution of PLGA 502H;

(b) Dropwise addition of the organic phase to 4 mL of 5% PVA solution, sonication of the mixture three times for 10 seconds each time, followed by the dropwise addition of the polymer/PVA solution to 196 mL of a 5% PVA solution in water; and

(c) dropwise addition of the organic phase to 4 mL of 5% PVA solution while using a homogenizer at 18000 rpm, followed by dropwise addition of the polymer/PVA solution to 196 mL of 5% PVA.

In procedure (b), the amplitude of the sonicator was varied between batches to observe the effect of amplitude on the release profiles. The amplitude was set at 15%, 30%, and 38%.

FIG. 2 shows the in vitro release profiles from the microparticles made using the modified procedures describes in methodologies (a)-(c) above. As is shown in the graph, the modified procedures had little effect on the duration of release of prednisolone acetate.

### Example 3

### In Vitro and In Vivo Release Studies of Methotrexate-Loaded Microspheres

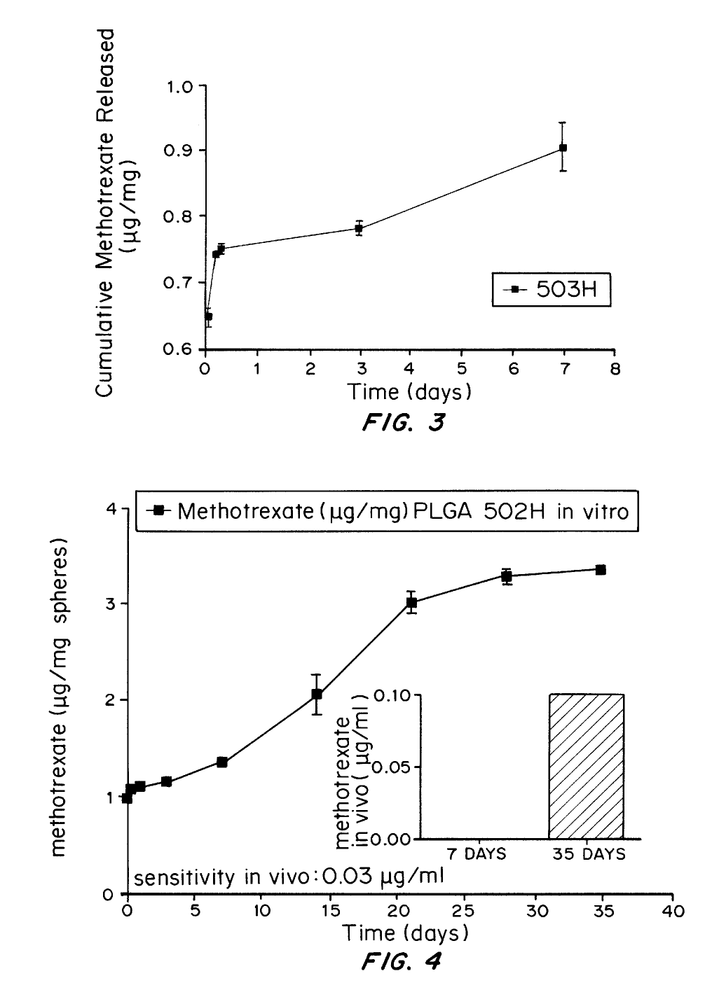

10% methotrexate-loaded microspheres were prepared as described in Example 1. The in vitro release study was conducted as described in Example 2. The results are shown in FIG. 3. FIG. 3 shows that the microspheres were still releasing methotrexate after 7 days from PLGA 503H microspheres.

FIG. 4 shows the in vivo release profile of methotrexate from PLGA 502H microspheres. The graph shows that an effective amount of methotrexate was released in vivo over at least 35 days.

### Example 4

### Preparation and in Vivo Release Studies of Triamcinolone-Loaded Microparticles

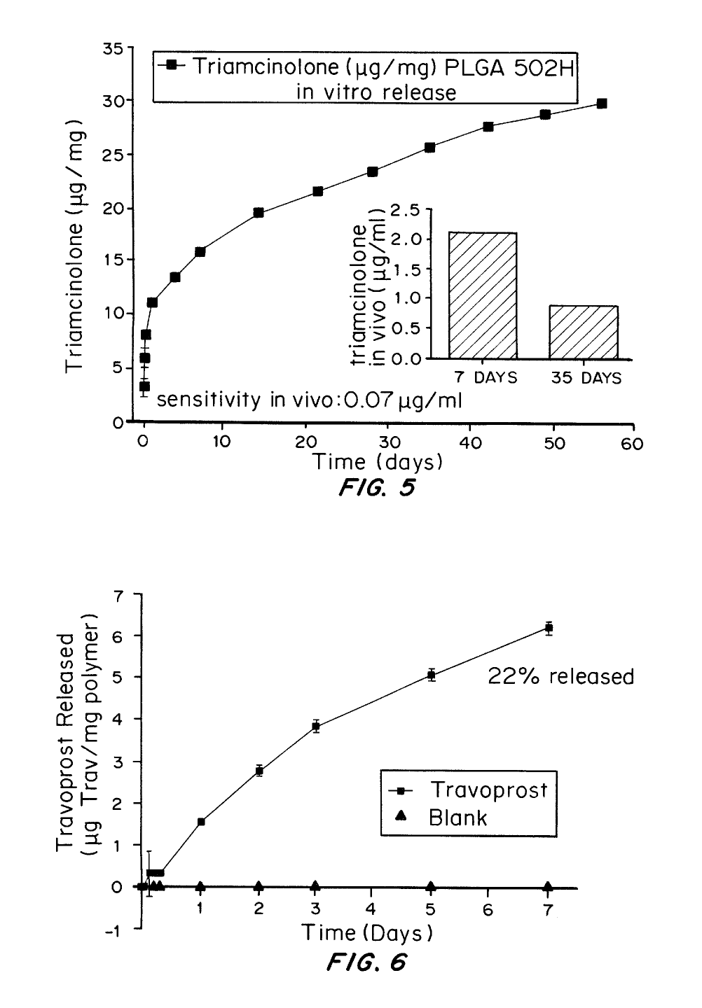

Triamcinolone microspheres were prepared using a water-in-oil-in-water (w/o/w) double emulsion solvent evaporation technique. FIG. 5 shows the in vitro release profile for triamcinolone-loaded microspheres. The microspheres provide a burst release over the first one to two days followed by linear release of an effective amount of triamcinolone over a period of about 60 days.

### Example 5

### Preparation and In Vitro Release Studies of Travoprost-Loaded Microspheres

Preparation of Microspheres

100 mg of PLGA 503H was dissolved in a 4:1 ratio of trifluoroethanol (TFE) and dichloromethane (DCM) (2.5 ml total). Travosprost is a liquid at room temperature. 1 mg of Travoprost was mixed with 50 μl of ethanol. The travoprost solution was added dropwise to the polymer solution and vortexed. The resulting solution was added dropwise to 100 ml of a 5% PVA solution while stirring. The solution was stirred for 3 hours to harden the microspheres, and the microspheres were collected, washed, and lyophilized.

In Vitro Release Studies

The in vitro release profile was measured using the procedure in Example 2. The results are shown in FIG. 6. The PLGA 503H-loaded microspheres release drug over a period of at least 7 days in a linear manner. At day 7, approximately 22% of the drug had been released.

### Example 6

### Preparation and In Vitro Release Studies of AG1478-Loaded Microspheres

Solid-in-Oil-in-Water Single Emulsion Technique

5 mg of AG1478 was suspended in 500 μL of dimethyl sulfoxide (DMSO). This suspension was added to 200 mg of PLGA 503H dissolved in 2 mL of dichloromethane (DCM) with vortexing (setting 5 on the vortex genie). The resulting suspension was added to 4 mL of 5% PVA (polyvinyl alcohol) while vortexing (set at 10). The resulting emulsion was added dropwise to 100 mL of 5% PVA and stirred for 3 hours. The spheres were collected by centrifugation and washed three times in MilliQ water. The microspheres were flash frozen and lyophilized. The resulting powder was stored at −20° C. until needed.

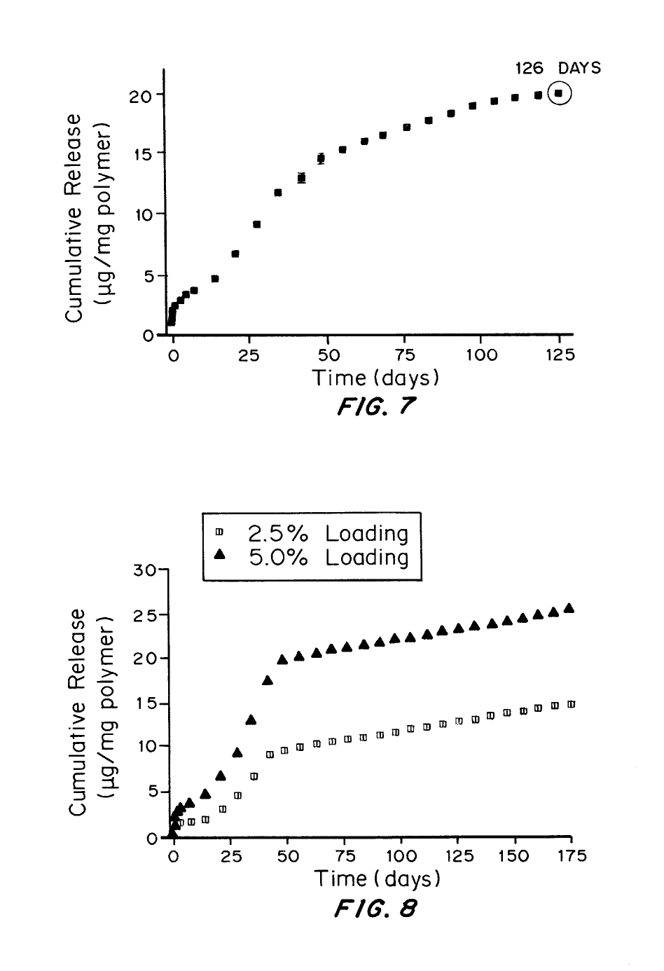

The in vitro release profile for microspheres prepared using the single emulsion technique is shown in FIG. 7. The microspheres released an effective amount of AG1478 in a linear manner for at least 126 days.

The rate of release is also influenced the percent loading of drug. FIG. 8 shows the release profile from microspheres prepared from PLGA 503H using via oil-in-water emulsion containing 2.5% and 5% AG1478. The microspheres loaded with 5% AG1478 released a greater amount of drug at a given time point than microspheres loaded with 2.5% AG1478.

Oil-in-Water Single Emulsion Technique

500 μL of DMSO was added to 5 mg of AG1478 and heated to 37° C. in a water bath to dissolve the AG1478. This solution was added to 200 mg PLGA 503H in 2 mL of DCM while vortexing. The PLGA-AG1478 solution was added dropwise to 4 mL of 5% PVA while vortexing. This emulsion was added dropwise to 100 mL of 5% PVA and stirred for 3H. The microspheres were collected and stored as described above.

Cosolvent Techniques

500 μL dimethyl sulfoxide (DMSO) was added to 5 mg of AG1478 and heated to 37° C. to dissolve the AG1478. This solution was added to 200 mg of PLGA 503H dissolved in 2.5 mL of DCM solvent and trifluoroethanol (TFE) cosolvent at a ratio of 1:5 (DCM:TFE, v/v). The PLGA-AG1478 solution was added to 4 mL of 5% PVA (polyvinylalcohol) while vortexing. The resulting emulsion was added dropwise to 100 mL of 5% PVA and stirred for 3 hours to harden the spheres. The spheres were collected, washed with deionized water, and stored at −20° C.

Loading of AG1478 Microspheres

Microsphere loading was dependent upon the single emulsion technique utilized to prepare each batch. Microspheres prepared using the o/w techniques resulted in higher loading, when compared to the s/o/w technique (15.1 μm/mg compared to 9.30 μg/mg). Furthermore, modifying the o/w emulsion with the addition of a co-solvent resulted in an approximate 38% increase in loading compared to o/w without a co-solvent (20.9 μg/mg and 15.1 μg/mg, respectively) and an approximate 125% increase in loading compared to s/o/w (9.30 μg/mg). The encapsulation efficiency for the three techniques was 29%, 55%, and 65%, respectively. The results suggest that it may be possible to titrate the loading of AG1478 in PLGA to achieve appropriate therapeutic doses (e.g. 10 mM).

Microsphere Sizing

The volume-weighted mean diameters of microspheres fabricated using the s/o/w and o/w emulsion were almost identical, 19.3±8.18 mm and 20.7±7.93 mm, respectively (mean±SD). However, the mean diameter of microspheres fabricated using the o/w emulsion with co-solvent was notably smaller (2.56±1.90 mm). SEM images confirmed the results obtained from the Coulter Multisizer and revealed microspheres that were heterogeneous in size with minimal aggregation and smooth surfaces.

Release of AG1478

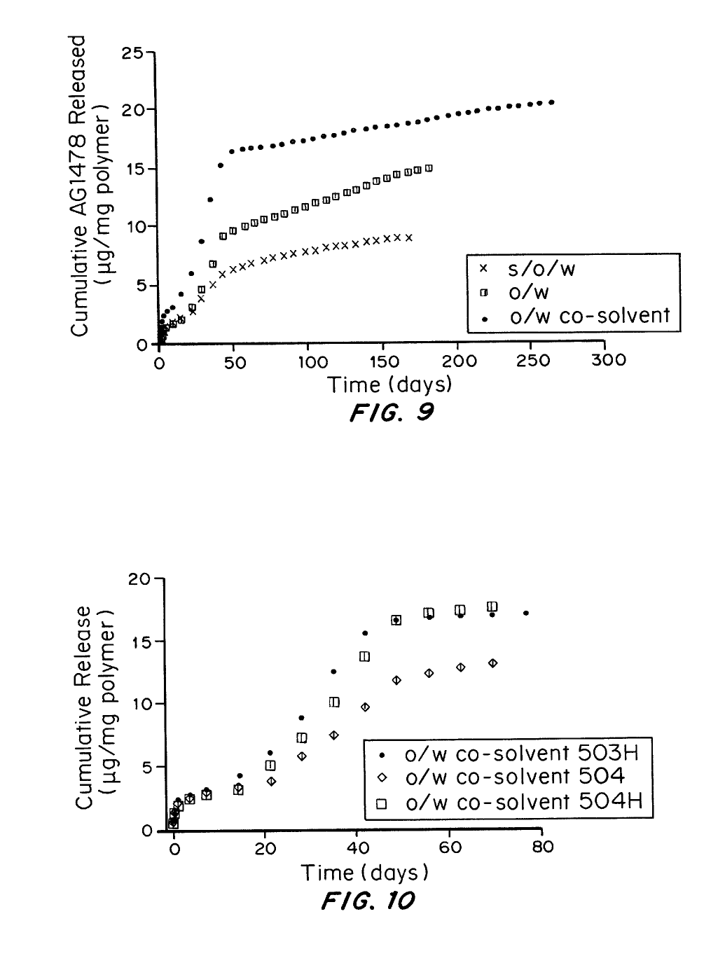

The in vitro release profiles for the microspheres prepared by three different processes, s/o/w, o/w, and o/w cosolvent are shown in FIG. 9. The use of a co-solvent results in a higher loading of drug and release of a great amount of drug at a given time point compared to microspheres prepared using oil-in-water emulsion techniques. All formulations of microspheres sustained release for at least 6 months. Further, microspheres prepared using the cosolvent technique released an effective amount of drug over at least 250-270 days, e.g. 266 days.

As expected, when comparing emulsion techniques, the amount of released drug was directly related to the amount loaded. The release kinetics of microspheres from all three formulations followed a typical triphasic release for PLGA microspheres, with an initial burst, followed by a lag phase and a secondary apparent-zero-order phase. However, the o/w emulsion with co-solvent resulted in about 1.7-times more AG1478 released after the first day when compared to the s/o/w and o/w emulsions. Comparing the percentage of total AG1478 released after 1 day revealed that the increase in the initial burst of AG1478 from microspheres prepared using the o/w emulsion with co-solvent (11.1±0.173%) was indeed significantly different to the amount of AG1478 released from microspheres prepared using the s/o/w emulsion (p50.001) and the o/w emulsion (p50.01) techniques (s/o/w=15.3±0.936% and o/w=8.05±0.248%, mean±SD).

FIG. 10 shows the release profile of AG1478 from microspheres prepared from PLGA 503H, PLGA 504, and PLGA 504H. The microspheres exhibit similar release profiles over the first 20 days. However, over the next 60 days, release of AG1478 was greater from the microspheres prepared from PLGA 503H and PLGA 504H. This likely due to the fact that the poorly water soluble AG1478 associates more strongly with the less hydrophilic PLGA 504 than with the more hydrophilic PLGA 503H and 504H.

FIG. 11 shows the release profile of AG1478 from microspheres prepared from PLGA 504 and PLGA 504H. The microspheres exhibited similar release profiles over the first 20 days. However, over the next 60 days, release was greater from the PLGA 504H microspheres. This likely due to the fact that the poorly water soluble AG1478 associates more strongly with the less hydrophilic PLGA 504 than with the more hydrophilic PLGA 504H.

Biological Activity of AG1478

To determine bioactivity of AG1478, supernatants were tested from release time points on Fisher rat 3T3 fibroblasts (FR3T3) and human A431 epithelial carcinoma cells (American Type Culture Collection, ATCC, Manassas, Va.). FR3T3 and A431 cells were maintained in high glucose DMEM supplemented with 10% fetal bovine serum and 1% antibiotic-antimycoctic (penicillin-streptomycin-amphoteticin B), at 37° C., 5% CO2.

Before bioactivity was assessed, the responsiveness of the two cell lines to AG1478 was determined. Cells were grown to 90% confluencey and serum-starved overnight in Opti-MEM. AG1478 was added to cells at concentrations ranging from 0-40 mM (about 0-12.6 mμ/mL) for 30 min at 37° C., 5% CO2. After 30 min, EGF (100 ng/mL) was added to the cells for 2 min. Cells were then collected and lysed in buffer (50 mM Tris-HCl, 100 mM NaCl, 5 mM EDTA, IX Complete EDTA-free Protease Inhibitor, 1 mM Na3VO4).

Protein concentration from cell lysates was determined using the Bio-RAD Protein Assay reagent and concentrated to at least 1-3 mg/mL total protein. Ten or twenty micrograms of protein in sample buffer (150 mM Tris-HCl, 6% SDS, 30% glycerol, 25% 2-mercaptoethanol, 0.0001% bromophenol blue) were added to each lane on reducing 6% SDSpolyacrylamide gels and transferred electrophoretically to nitrocellulose. The blots were then blocked with 5% non-fat milk in Tris Buffered Saline and Tween and incubated with rabbit polyclonal antibody against EGFR (Cell Signaling Technology, Danvers, Mass., working dilution 1:500, IgG) or mouse monoclonal antibody against phosphorylated-EGFR (Cell Signaling Technology, Danvers, Mass., Tyr 1068, working dilution 1:500, IgG). Blots were also incubated with mouse monoclonal antibody against alpha-tubulin (Invitrogen, Carlsbad, Calif., working dilution 1:2000, IgG) as a loading control. Blots were then incubated with the appropriate AP conjugated secondary antibodies (working dilutions 1:2000) and developed using the 1-Step NBT/BCIP reagent in AP buffer (100 mM Tris-HCl, 100 mM NaCl, 5 mM MgCl₂*6H2O). From these blots, 1050 values of AG1478 were determined for each cell line.

Bioactivity was assessed by adding sterile supernatants from day 35 of release from one batch of AG1478 microspheres at concentrations equal to the 1050 value to serum-starved cells for 30 min. After 30 min, EGF (100 ng/mL) was added to the cells for 2 min. Cells were collected and protein was concentrated as described above. Protein from cell lysates were electrophoretically separated on reducing 6% SDS-polyacrylamide gels and blotted as described above.

Western blot analysis demonstrated that the encapsulated inhibitor retained bioactivity and that it was as effective as a non-encapsulated inhibitor in cells expressing EGFR at normal levels (89±4.5% and 89±2.7%, respectively).

To determine whether the encapsulated AG1478 would also be effective against cells with EGFR over-expression, A431 cells were tested as above with encapsulated and non-encapsulated AG1478 at the appropriate 1050 value (10.5 nM, 3.3 ng/mL). The western blot results showed that the activity of the released encapsulated inhibitor was not significantly different from the activity of the non-encapsulated inhibitor. A significant reduction in the level of EGFR tyrosine phosphorylation was observed for encapsulated inhibitor compared to untreated cells.

In addition, this study also tested encapsulated AG1478 from later release time points and confirmed its bioactivity in FR3T3 cells, demonstrating that this encapsulation system remains effective until the polymer has completely degraded and all drug has been released.

### Example 7

### AG1478 Promotes Optic Nerve Regeneration

Glaucoma is a group of neurodegenerative eye diseases typified by structural damage to the optic nerve that causes selective death of retinal ganglion cells (RGCs) leading to blindness. Early diagnosis and intervention can slow the progression of this disease; however current clinical therapeutic options fail to rescue or repair damaged RGCs. Recent work suggests that administration of the small-molecule epidermal growth factor receptor (EGFR) tyrosine kinase inhibitor AG1478 promotes robust nerve regeneration and RGC survival. Adverse side effects of oral and systemic delivery of AG1478, make a single-dose, localized, minimally invasive administration of the treatment advantageous. In addition, pre-clinical studies have shown that sustained levels of AG1478 must be maintained to effectively inhibit EGFR.

Therefore AG1478 was encapsulated in poly(lactic-co-glycolic acid) (PLGA) microspheres. It was hypothesized that local and sustained delivery of AG1478 would lead to increased regeneration in the injured optic nerve.

Methods: PLGA (50311) microspheres encapsulating AG1478, Coumarin-6—for tracking purposes—or no drug (blanks; control), were fabricated using a single emulsion technique with a co-solvent formulation of either 1:5 or 1:4 (dichloromethane:trifluoroethanol, DCM:TFE). Microsphere size was ascertained using a Multisizer™ 3 Coulter Counter® and confirmed visually via SEM. Loading and release of AG1478 microspheres was determined using UV-Vis at 330 nm. To confirm bioactivity of AG1478 after encapsulation, encapsulated and non-encapsulated AG1478 was added to FR3T3 cells in the presence of EGF. Cells were collected, lysed, and electrophoretically separated on reducing 6% SDS-polyacrylamide gels and then blotted for EGFR, phospho-EGFR, and α-Tubulin. Relative bioactivity was determined using the gray mean value for each phospho-EGFR band.

After fabrication of microspheres and confirmation of bioactivity, AG1478 microspheres were administered in a rat optic nerve crush injury model to ascertain the effects on nerve regeneration. Briefly, the optic nerve was crushed for 10 s and 5 mins later a 5 μL volume of AG1478 microspheres suspended in 1×DPBS were injected into the sub-tenon space. At 1, 2, 4, and 7-week time points animals were sacrificed and the globe and nerve were dissected, cryo-sectioned and immunostained for markers of regeneration (e.g., GAP-43), gliosis (e.g., GFAP), and immune reaction (e.g., CD68).

Results:

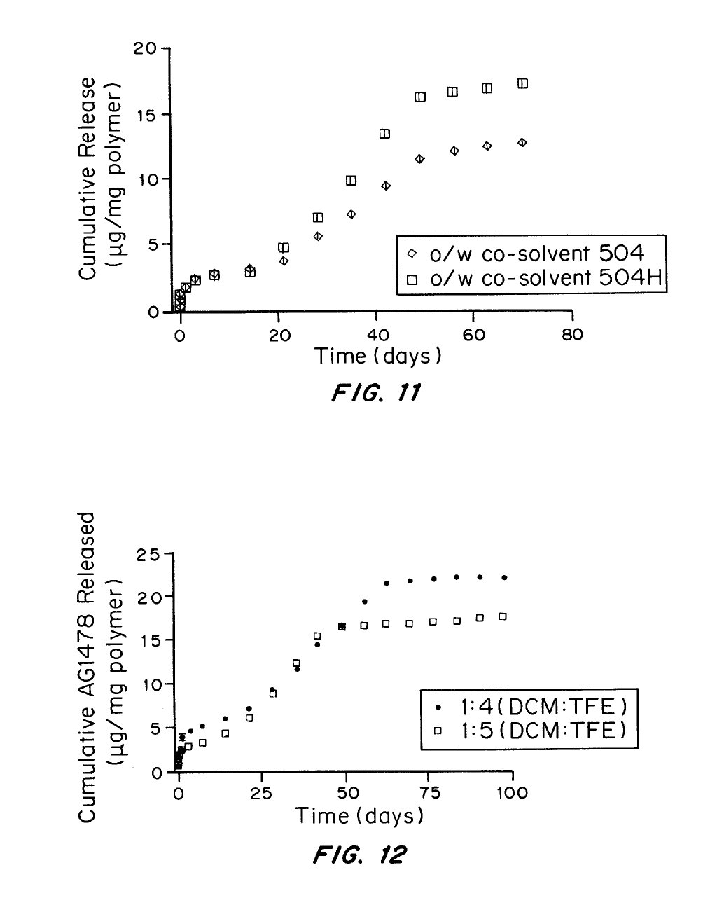

Microspheres were on average 2.56±1.90 μm in size. By increasing the ratio of DCM:TFE from 1:5 to 1:4 we were able to increase encapsulation from 65% (21 μg/mg polymer) to 76% (22 μg/mg polymer), respectively (FIG. 12). Based on western blot analysis of activated EGFR, encapsulated AG1478 displayed the same bioactivity in vitro as non-encapsulated AG1478 (89±2.7% and 89±4.5%, respectively; mean±SEM). Coumarin-6 microspheres were injected into the sub-tenon space to determine the location and persistence of microspheres. Coumarin-6 microspheres could be found proximal to the crush site as long as 7 weeks—the longest time point assayed—after injury. Administration of AG1478 and blank microspheres in vivo revealed significant differences in regeneration between the two groups. GAP-43 staining was higher in the optic nerve of animals that received AG1478 microspheres versus animals that received blank microspheres. In addition, regenerating fibers could be observed more than 1500 μm past the crush site. Analysis of GFAP and CD68 showed no differences between groups.

The data demonstrates that AG1478 can be encapsulated in PLGA microspheres and retain its bioactivity. It was found that by increasing the amount of water-miscible solvent in the co-solvent ratio, the encapsulation can be significantly increased. Using a sub-tenon injection, microspheres persist for up to 7 weeks and deposit on the optic, near the crush site. Furthermore, administration of AG1478 microspheres greatly enhanced nerve regeneration compared to animals that received blank microspheres. These findings indicate that local and sustained delivery of AG1478 or other epidermal growth factor receptor antagonist using PLGA microspheres can be used for promoting neural regeneration for the treatment of glaucoma and CNS nerve injury more broadly.

We claim:

1. A biodegradable injectable polymeric microparticulate pharmaceutical composition for delivery of a poorly water-soluble active agent,
wherein the biodegradable polymeric microparticles have a diameter between one and twenty-five microns, comprise a biodegradable polymer and between one and 50 weight percent active agent dispersed therein,
wherein the hydrophobicity of the polymer forming the microparticles corresponds to the hydrophobicity of the active agent to be released, the hydrophobicity and charge of the polymer are selected to optimize percent loading of the active agent relative to a particle where the hydrophobicity is not optimized, and the molecular weight and monomer composition result in release of an effective amount of the active agent over a period of time of at least 60 days equivalent to administration of the active agent via the same route of administration in the absence of microparticles.

2. The composition of claim 1, wherein the microparticles are formed from one or more polymers selected from the group consisting of poly(lactic-co-glycolic) acid (PLGA), a blend of PLGA and polylactic acid (PLA).

3. The composition of claim 1, wherein the one or more active agents are selected from the group consisting of active agents that lower intraocular pressure, antibiotics, steroids, growth factors, chemotherapeutic agents, and combinations thereof.

4. The composition of claim 3, wherein the active agent that lowers intraocular pressures is selected from the group consisting travoprost, bimatoprost, latanoprost, and combinations thereof.

5. The composition of claim 3, wherein the antibiotic is selected from the group consisting of cephaloridine, cefamandole, cefamandole nafate, cefazolin, cefoxitin, cephacetrile sodium, cephalexin, cephaloglycin, cephalosporin C, cephalothin, cafcillin, cephamycins, cephapirin sodium, cephradine, penicillin BT, penicillin N, penicillin O, phenethicillin potassium, pivampic ulin, amoxicillin, ampicillin, cefatoxin, cefotaxime, moxalactam, cefoperazone, cefsulodin, ceflizoxime, ceforanide, cefiaxone, ceftazidime, thienamycin, N-formimidoyl thienamycin, clavulanic acid, penemcarboxylic acid, piperacillin, sulbactam, cyclosporin, and combinations thereof.

6. The composition of claim 3, wherein the active agent is the growth factor inhibitor AG1478.

7. The composition of claim 3, wherein the steroid is selected from the group consisting of prednisolone acetate, triamcinolone, prednisolone, hydrocortisone, hydrocortisone acetate, hydrocortisone valerate, vidarabine, fluorometholone, fluocinolone acetonide, triamcinolone acetonide, dexamethasone, dexamethasone acetate, and combinations thereof.

8. The composition of claim 4, wherein the one or more active agents is travoprost or a pharmaceutically acceptable salt thereof.

9. The composition of claim 1, wherein the percent loading of active agent is between 5 and 30 weight percent.

10. The composition of claim 1, wherein the polymer is PLGA having a molecular weight in the range from about 10 kD to about 80 kD.

11. The composition of claim 1 wherein the period of release is 90 days or greater in vivo.

12. The composition of claim 1 wherein the polymer is treated to increase the number of carboxyl groups.

13. The composition of claim 12 wherein the polymer is PLGA.

14. The composition of claim 1, wherein the composition further comprises one or more pharmaceutically acceptable excipients.

15. A method for administering a poorly water soluble active agent, comprising administering to a site in an individual a biodegradable polymeric microparticulate pharmaceutical composition for delivery of the active agent,
wherein the biodegradable polymeric microparticles have a diameter between one and twenty-five microns, comprise a biodegradable polymer and between one and 50 weight percent active agent dispersed therein,
wherein the hydrophobicity of the polymer forming the microparticles corresponds to the hydrophobicity of the active agent to be released, the hydrophobic and charge of the polymer are selected to optimize percent loading of the active agent relative to a particle where the hydrophobicity is not optimized, and the molecular weight and monomer composition result in release of an effective amount of the active agent over a period of time of at least 60 days equivalent to administration of the active agent via the same route of administration in the absence of microparticles.

16. The method of claim 15 for delivering drug to the eye, comprising administering the microparticles to the eye.

17. The method of claim 15, wherein the microparticles are fainted from poly(lactic-co-glycolic) acid (PLGA) or a blend of PLGA and polylactic acid (PLA).

18. The method of claim 15, wherein the one or more active agents are selected from the group consisting of active agents that lower intraocular pressure, antibiotics, steroids, growth factors, chemotherapeutic agents, and combinations thereof.

19. The method of claim 18, wherein the active agent that lowers intraocular pressures is selected from the group consisting of travoprost, bimatoprost, latanoprost, and combinations thereof.

20. The method of claim 18, wherein the antibiotic is selected from the group consisting of cephaloridine, cefamandole, cefamandole nafate, cefazolin, cefoxitin, cephacetrile sodium, cephalexin, cephaloglycin, cephalosporin C, cephalothin, cafcillin, cephamycins, cephapirin sodium, cephradine, penicillin BT, penicillin N, penicillin O, phenethicillin potassium, pivampic ulin, amoxicillin, ampicillin, cefatoxin, cefotaxime, moxalactam, cefoperazone, cefsulodin, ceflizoxime, ceforanide, cefiaxone, ceftazidime, thienamycin, N-formimidoyl thienamycin, clavulanic acid, penemcarboxylic acid, piperacillin, sulbactam, cyclosporins, and combinations thereof.

21. The method of claim 18, wherein the active agent is the growth factor inhibitor AG1478.

22. The method of claim 18, wherein the steroid is selected from the group consisting of prednisolone acetate, triamcinolone, prednisolone, hydrocortisone, hydrocortisone acetate, hydrocortisone valerate, vidarabine, fluorometholone, fluocinolone acetonide, triamcinolone acetonide, dexamethasone, dexamethasone acetate, and combinations thereof.

23. The method of claim 19, wherein the one or more active agents is travoprost or a pharmaceutically acceptable salt thereof.

24. The composition of claim 15 wherein the polymer is a carboxylated PLGA and the percent loading of drug is from 5 to 30% by weight.

25. The method of claim 24, wherein the polymer is a PLGA with a molecular weight from about 10 kD to about 80 kD.

26. The method of claim 15, wherein the composition is administered by injection.

27. The method of claim 26, wherein the composition is administered subconjunctivally.

28. A kit comprising the composition of claim 1.

29. The kit of claim 28, wherein the kit further comprises instructions for preparing and/or administering the composition, optionally comprising a needle and syringe for administering the composition.

30. The kit of claim 28, wherein the microparticles and the carrier are stored in the same container or in separate containers.

31. The kit of claim 30, wherein the container is selected from the group consisting of sterile vials, jars, sealed ampules, and combinations thereof.
